(19) United States
(12) Patent Application Publication
(10) Pub. No.: US 2009/0234417 A1
(43) Pub. Date: Sep. 17, 2009

(54) Methods And Apparatus For The Treatment Of Metabolic Disorders

(75) Inventors: James R. Pastena, Succasunna, NJ (US); Joseph P. Errico, Green Brook, NJ (US); Steven Mendez, Chester, NJ (US); Hecheng Hu, Cedar Grove, NJ (US); Bruce Simon, Mountain Lakes, NJ (US)

Correspondence Address: ELECTROCORE INC., 51 GILBRALTAR DRIVE, SUITE 2F, POWER MILL PLAZA, MORRIS PLAINS, NJ, 07050-1254, US

(73) Assignee: ElectroCore, Inc., Morris Plains, NJ (US)

(21) Appl. No.: 12/432,946

(22) Filed: Apr. 30, 2009

Related U.S. Application Data
(63) Continuation-in-part of application No. 11/555,142, filed on Oct. 31, 2006.
(60) Provisional application No. 61/050,599, filed on May 5, 2008.
(60) Provisional application No. 60/736,001, filed on Nov. 10, 2005.

(51) Int. Cl. A61N 1/36; A61F 7/12; A61H 1/00

(52) U.S. Cl. 607/40; 607/113; 601/46

(57) ABSTRACT

Systems and methods are disclosed for treatment of metabolic disorders such as type 2 diabetes and obesity by stimulation of the small intestine to modulate hormone production. Methods include applying an appropriate signal to a region of the small intestine to modulate hormone production. The method may involve applying a signal to the nerves that innervate the small intestine. Devices for delivering the signal are disclosed.

### CROSS REFERENCE TO RELATED APPLICATIONS

This application claims the benefit of commonly assigned co-pending U.S. Provisional Patent Application Ser. No. 61/050,599, filed May 5, 2008; and this application is a continuation-in-part of commonly assigned co-pending U.S. patent application Ser. No. 11/555,142 filed Oct. 31, 2006, which claims the benefit of U.S. Provisional Patent Application Ser. No. 60/736,001, filed Nov. 10, 2005, the entire disclosures of which are hereby incorporated by reference. This application is also related to commonly assigned co-pending U.S. patent Ser. Nos. 11/555,170, 11/592,095, 11/591,340, 11/591,768, 11/754,522, 11/735,709 and 12/246,605, the complete disclosures of which are incorporated herein by reference for all purposes.

### BACKGROUND OF THE INVENTION

The present invention relates to the field of delivery of electrical impulses (and/or fields) to bodily tissues for therapeutic purposes, and more specifically to devices and methods for modulating hormones in the digestive tract to treat metabolic disorders.

The use of electrical stimulation has been well known in the art for nearly two thousand years. Roman physicians are reported to have used electric eels for treating headaches and pain associated with gout. In 1760, John Wesley used the primitive rudimentary electrical device, the Leyden Jar, was applied to therapeutic purposes hoping to shock patients suffering from paralysis, convulsions, seizures, headaches, angina, and sciatica.

It was not until Luigi Galvani, in 1791, that a disciplined study of the effects of electricity on muscles and nerves was done in a scientifically rigorous manner. In 1793, Alessandro Volta furthered this work when he reported that muscle contraction could be forced to occur when an electrified metal was placed in the vicinity of a motor nerve and the muscle innervated by that nerve.

One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed. Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

The application of this electrical stimulation to the nervous system for other medical applications, of course, includes electroshock therapy for mental illness, such as for schizophrenia and depression. Early brute force attempts to apply voltage across the skull have, thankfully, evolved to the point where leads are being implanted into very specifically mapped regions of the brain, so that precise amounts of electricity can be applied far more effectively, and with far fewer complications (see U.S. Pat. No. 6,871,098 to Nuttin, et al., the disclosure of which is incorporated herein by reference).

The applications for deep brain stimulation go beyond simply mental illness of a behavioral nature, but also extend to degenerative motor dysfunctions associated with brain-based pathologies, such as Parkinsons disease and essential tremor (see, for example, Meadows, et al. U.S. Pat. No. 6,920,359, the teachings and specification of which are incorporated herein by reference). Certain facial and body pain can be treated by applying electrical stimulation to the surface of the brain as well, for example, see U.S. Pat. No. 6,735,475 to Whitehurst, et al., the disclosure of which is incorporated herein by reference.

Another application of electrical stimulation of nerves has been the treatment of radiating pain in the lower extremities by means of stimulation of the sacral nerve roots at the bottom of the spinal cord (see U.S. Pat. No. 6,871,099 to Whitehurst, et al., the disclosure of which is incorporated herein by reference).

Just as the stimulation of the brain can be used to treat pain and motor function pathologies in the body, nerve stimulation in the periphery can be used to affect the behavior of patients. For example, treatments for depression and overeating have been utilized with varying degrees of reported success within the past decade.

It is well documented that excessive body fat, overeating and obesity are associated with a myriad of gastrointestinal diseases and conditions. Efforts to treat these diseases and conditions have been principally pharmaceutical and surgical, with varying degrees of success. With the advent of laparoscopic surgical techniques, surgeries relating to the gastrointestinal tract have increased. For example, cholecystectomies (removals of the gallbladder) are being performed at the rate of over five hundred thousand per year in the United States alone. Gastric bypass surgery has become routine as a treatment for obesity. Recently, the surgical removal of the duodenum has become an increasingly common surgical gastric bypass technique, with 177,600 such operations performed in the United States last year. However, such surgeries often have unwanted side effects and the risks associated with surgery are well known.

A number of electrical devices and processes are taught in the art for attempting to control an individual's food intake and/or various aspects of the digestive process in an effort to treat eating or digestive disorders. Some prior art references focus on the movement of food. Chen, et al., U.S. Pat. No. 5,690,691, discloses a gastric pacemaker implantable in the gastro-intestinal tract to deliver a phased electrical stimulation to pace peristalsis to enhance or accelerate peristaltic movement through the gastric tract or to attenuate the peristaltic movement to treat such conditions eating disorders or diarrhea. Likewise, Terry, Jr., et al., U.S. Pat. No. 5,540,730, discloses an apparatus and method of treating motility disorders by selectively stimulating a patient's vagus nerve to modulate electrical activity of the nerve and to thereby cause a selective release or suppression of excitatory or inhibitory transmitters. One embodiment employs the manual or automatic activation of an implanted device for selective modulation. Similarly, Cigaina, U.S. Pat. No. 5,423,872, discloses a process and device for treating obesity and syndromes related to motor disorders of the stomach by altering the natural gastric motility of a patient by electrical stimulation to prevent emptying or to slow down food transit.

U.S. Patent Application Number 20050222637, to Chen, entitled Tachygastrial Electrical Stimulation, which is incorporated by reference herein, discloses treating obesity with electrical pulses to artificially alter the natural gastric motility of the patient to prevent the emptying of or to slow down gastric transit through the stomach. This increases the feeling of satiety and/or accelerates intestinal transit to reduce absorption time within the intestinal tract.

Other prior art references focus on sensory aspects of food consumption. Zikria, U.S. Pat. No. 6,564,101, discloses a system for controlling a patient's appetite using an electrical signal controller that sends electrical signals to the fundus of the patient's stomach, wherein the controller generates substantially continuous low voltage stimulation with varying periodicity as determined by the individual's specific physiology, anatomy and/or psychology.

Wernicke, et al., U.S. Pat. No. 5,188,104 (“'104”), which is incorporated by reference herein, discloses a method and apparatus of using electrical stimulation of the vagus nerve to treat patients with compulsive eating disorders. The '104 patent proposes “detecting a preselected event indicative of an imminent need for treatment of the specific eating disorder of interest, and responding to the detected occurrence of the preselected event by applying a predetermined stimulating signal to the patient's vagus nerve appropriate to alleviate the effect of the eating disorder of interest.”

Several recent clinical studies have demonstrated that gastric bypass surgical procedures for treating obesity, including Roux-en-Y, bilio-pancreatic diversion and duodenum exclusion, show a rapid and remarkable reduction in clinical symptoms of diabetes including normalization of glucose and insulin levels. These effects occur before any changes in obesity and suggest that the duodenum may secrete molecular signals that cause insulin resistance. Supportive data has also been demonstrated in rat models of diabetes. See, Rubino and Marescaux, Annals of Surgery, 239 No. 1, 1-11 (January 2004), the entirety of which is incorporated herein by reference.

In view of the foregoing there is a need in the art for techniques involving the modulation of gastrointestinal hormones to effect treatment of metabolic disorders and conditions without drugs or surgery.

### SUMMARY OF THE INVENTION

The present invention provides systems, apparatus and methods for selectively applying electrical energy to body tissue. In particular, biophysical stimulation methods and devices are provided to restore normal insulin/glucose metabolism without the need for gastric bypass surgery.

In one aspect of the present invention, a method for treating or preventing type 2 diabetes includes applying energy to at least one region of the small intestine of the patient to modulate the hormone releasing activity of the cells of that region. The target region of the small intestine is preferably on the outer or inner surface, within the walls, or within the lumen, of one or more of the pyloric sphincter, the duodenum or the jejunum. The energy is sufficient to at least partially inhibit or offset the production of molecular signals or hormones that cause insulin resistance in certain patients. Thus, similar to the more invasive gastric bypass surgical procedures, the present invention can be used to help normalize glucose and insulin levels in patients suffering from type 2 diabetes without the adverse side-effects of these surgical procedures.

In one embodiment, the energy is transmitted to the submucosa region of the small intestinal wall. This region is responsible for sensing the environment within the lumen to control epithelial cell function. In this embodiment, sufficient energy is applied to the submocosa region to modulate the signals generated from this region, thereby controlling the release of hormones that may cause insulin resistance in certain patients.

In another embodiment, the energy is transmitted to the epithelial cells lining the lumen of the small intestine. These cells are responsible for actually secreting gastrointestinal hormones into the lumen of the GI tract. In this embodiment, sufficient energy is applied to these cells to modulate their production of hormones that may cause insulin resistance in certain patients.

The energy transmitted to the target region may be in the form of electrical, vibrational, mechanical or temperature.

In one embodiment, an electrical impulse is applied to one or more electrode(s) positioned at or within close proximity to the target region. The mechanisms by which the appropriate stimulation is applied to the target tissue can include positioning the distal ends of an electrical lead or leads in the vicinity of a region of the small intestine, either within or on the outside or inside of the intestinal wall. The electrodes are preferably powered by an internal stimulator or through an external stimulator that is inductively coupled to a receiver in the body. The electric field generated at the distal tip of the lead creates a field of effect that permeates the target tissue and cause the modulation of hormone release in the target region.

Alternatively, the electrode(s) may be positioned on the outer surface of the patient's skin such that the electrical impulse is delivered non-invasively through the patient's skin to the target region. In this embodiment, non-invasive stimulation can be effected with pulsed electromagnetic fields or through capacitively coupled electrodes on the skin.

In a first embodiment, the method of the present invention includes driving an excitatory and/or non-excitatory signal to the cells of the enteric endocrine system that control hormone production. In a second embodiment, the method includes driving an excitatory and/or non-excitatory signal to the nerve plexus and/or surrounding nerve tissues innervating the enteric endocrine system. In both embodiments, the methods of the present invention modulate hormones and neurotransmitters including but not limited to: secretin, cholecystokinin, gastrin, gastrin inhibitory protein, nitric oxide, vasoactive intestinal peptide, glucagon-like peptide, peptide YY, ghrelin, motilin, and other incretins and “anti-incretins”.

In preferred embodiments, methods are provided wherein the modulation signals are applied in a manner that promotes or inhibits hormone secretion in the region of the small intestine with which the signal is associated. It shall be understood that the activation of such signals may be directed manually by the patient, or automatically through a feedback mechanism that recognizes and responds to a state of the small intestine. For example, the pH of the duodenum may be monitored, and when it is found to dip below or above a threshold level, the hormone secreting cells of the duodenum may be triggered to increase or decrease the secretion of cholecystokinin or secretin.

In another embodiment, one or more flexible, stent-like coil(s) are implanted in the lumen of the duodenum and/or another region of the small intestine. An external electromagnetic device (tuned to the resonant frequency of the coil) is used to heat the coil(s) several ° C. with a specific duty cycle to modulate the hormone releasing activity of the region in which the coil(s) are implanted to restore normal glucose metabolism.

In another embodiment, a mechanical signal is applied to the target region within the small intestine. A coil is placed within the target region and connected to an implanted mechanical actuator. Alternatively, a coil made of a temperature sensitive memory metal such as but not limited to nitinol is placed within the target region. An external device drives expansion and contraction of the coil by slight changes in temperature produced by an external electromagnetic field. Alternatively, the coil is connected to a direct current source which can modulate the pH of the duodenum and/or other regions of the small intestine through the release of acidic or basic Faradic Products.

In certain embodiments, the signal includes bursts of pulses repeated at a fixed frequency. The amplitude of each pulse will vary from 0.1 gauss to 50 gauss, preferably between about 10 to 20 gauss. The number of pulses/burst will range from 1 to 200, preferably between about 5 to 30 pulses/burst. The burst frequency ranges from 2 Hz to 100 Hz, preferably between about 5 to 50 Hz. The pulse duration varies from 10 μs to 10 ms, preferably between about 20 to 1000 μs. In a particularly preferred embodiment, the amplitude of the signal is 16 gauss, there are 20 pulses/burst, the bursts repeat at 15 Hz and during each pulse the magnetic field rises linearly to 16 gauss in 200 μs and then decays linearly to 0 in 25 μs.

In yet another embodiment, the method includes delivering a capacitively coupled (CC) electric field delivered via conducting electrodes placed on the outside or the inside of the sinusoidal voltage across the electrodes. The frequency of the signal may vary from 1 kHz to 100 kHz and may have a duty cycle from 1% to 100%. The amplitude of the signal is chosen so as to produce an electric field at the location of the device of from 0.1 to 100 mV/cm. In a preferred embodiment, the signal is a 60 kHz sine wave with amplitude of 20 mV/cm at the device.

In yet another embodiment, the method includes delivering an amplitude modulated radiofrequency signal with by an external coil, similar to that used in an MRI machine. In this embodiment, the device acts as an antenna. The carrier frequency of the signal is such that its' wavelength in the tissue is twice the length of the device. This will allow for maximal coupling of the signal to the device. For a typical device this corresponds to a carrier frequency of about 1-2 GHz. This carrier frequency may be modulated with waveforms as described above. In a preferred embodiment, the modulating waveform is a 5 ms long pulse repeating at 15 Hz.

In another embodiment, the device is made from a piezoelectric material. As intestinal pressure increases or decreases, the change in pressure produces a time varying potential difference across the device which stimulates a signal. Alternatively, the device may be coated with a thin electret material. The permanent electric field at the surface of the electret stimulates a signal.

In another embodiment, the device is coated with a material having a very high dielectric constant due to a high density of charged groups (similar to the glycocolyx surrounding a cell). As material flows past this layer, it generates a streaming potential which stimulates a signal.

In another embodiment, hormone secretion by the duodenum or other region of the small intestine is inhibited by thermal ablation of one or more regions of the lumen of the duodenum.

In another aspect of the invention, a method for treating obesity includes applying energy to at least one region of the small intestine of a patient to modulate hormone secretions. The energy is preferably sufficient to reduce the volume of bile flow from the common biliary duct of a patient. Reducing the flow of bile into the stomach reduces hunger sensations within the patient, thereby affecting weight loss in the patient. In a preferred embodiment, the method includes applying an electrical impulse either directly to the small intestine, such as the duodenum, jejunum or pylorus, or to nerve fibers associated with the hormone secreting cells in the enteric endocrine system.

The application of stimulation in the form of electrical, vibrational and/or temperature stimulation, to one or more regions of the small intestine to modulate the release of hormones is more completely described in the following detailed description of the invention, with reference to the drawings provided herewith, and in claims appended hereto.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 is a schematic view of a portion of the gastrointestinal tract of a patient;

FIG. 2 is a diagram of a typical mammalian digestive tube, illustrating the layers thereof;

FIG. 3 is a schematic view of a impulse generating device according to one or more embodiments of the present invention;

FIG. 4 is a graphical illustration of an electrical signal profile that may be used to treat disorders through hormonal modulation in accordance with one or more embodiments of the present invention; and

FIG. 5 is a schematic view of electrodes implanted in the duodenum of a patient according to certain embodiments of the present invention.

### DETAILED DESCRIPTION OF THE PREFERRED EMBODIMENTS

In the present invention, electrical energy is applied to a target region within a patient's body. The invention is particularly useful for applying electrical impulses that interact with the signals of one or more nerves, or muscles, to achieve a therapeutic result, such as the treatment of metabolic disorders (e.g., obesity and/or type II diabetes). For convenience, the remaining disclosure will be directed specifically to the treatment of nerves and muscles associated with the enteric endocrine system or the nerve plexus and/or surrounding nerve tissues innervating the enteric endocrine system, but it will be appreciated that the systems and methods of the present invention can be applied equally well to other tissues and nerves of the body, including but not limited to other parasympathetic nerves, sympathetic nerves, spinal or cranial nerves, e.g., optic nerve, facial nerves, vestibulocochlear nerves and the like.

Digestive function is affected by hormones produced in many endocrine glands, but the most profound control is exerted by hormones produced within the gastrointestinal tract. The gastrointestinal tract is the largest endocrine organ in the body and the endocrine cells within it are referred to collectively as the enteric endocrine system. Some of the principal enteric hormones are gastrin, which is secreted from the stomach and plays an important role in control of gastric acid secretion; cholecystokinin, a small intestinal hormone that stimulates secretion of pancreatic enzymes and bile, secretin, another hormone secreted from small intestinal epithelial cells, which stimulates secretion of a bicarbonate-rich fluids from the pancreas and liver; ghrelin, motilin, and gastric inhibitory polypeptide.

In contrast to endocrine glands like the anterior pituitary gland, in which essentially all cells produce hormones, the enteric endocrine system is diffuse: single hormone-secreting cells are scattered among other types of epithelial cells in the mucosa of the stomach and small intestine. For example, most of the epithelial cells in the stomach are dedicated to secreting mucus, hydrochloric acid or a proenzyme called pepsinogen into the lumen of the stomach. Scattered among these secretory epithelial cells are G cells, which are endocrine cells that synthesize and secrete the hormone gastrin. Being a hormone, gastrin is secreted into blood, not into the lumen of the stomach. Similarly, other hormones produced by the enteric endocrine system are synthesized and secreted by cells within the epithelium of the small intestine.

Cells in the enteric endocrine system secrete hormones in response to fairly specific stimuli and stop secreting their hormone when those stimuli are no longer present. In most cases these endocrine cells respond to changes in the environment within the lumen of the digestive tube. Because these cells are part of the epithelium, their apical border is in contact with the contents of the lumen, which allows them to continually “taste” or sample the lumenal environment and respond appropriately.

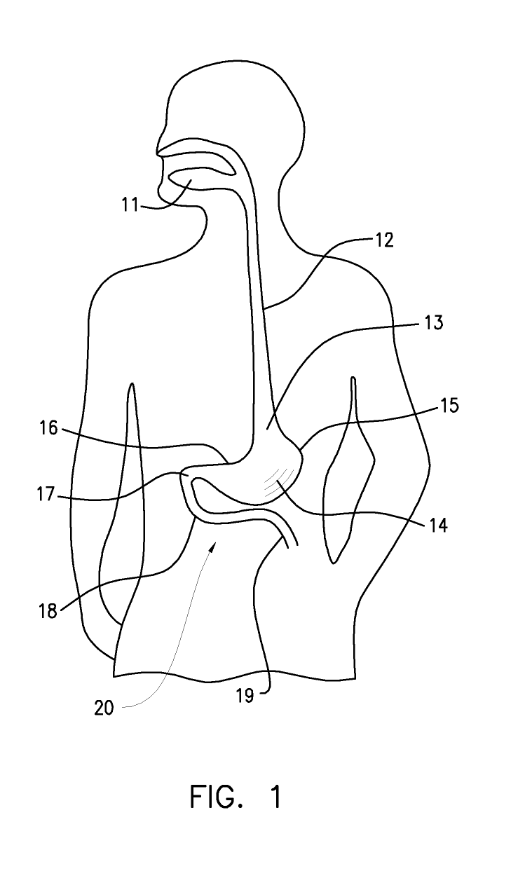

FIG. 1 is a schematic diagram of a portion of the gastrointestinal tract. The esophagus 12 terminates at the nose or mouth 11 at its superior end and at the stomach 14 at its inferior end. The stomach 14 is a generally contoured sac having a greater curvature 15 and a lesser curvature 16. Two smooth muscle valves, or sphincters, contain the contents of the stomach within the stomach upon ingestion. These smooth muscle valves are the esophageal sphincter 13, found in the cardiac region above the antrum cardiacum, and the pyloric sphincter 17 disposed between the stomach 14 and the small intestine 20. Pyloric sphincter 17 is a strong ring of smooth muscle at the end of the pyloric canal that functions to help regulate the passage of chyme from stomach 14 to the duodenum 18. The stomach empties from the pylorus 17 to the duodenum 18, which is the upper or proximal portion of the small intestines. Gastric contents pass through the duodenum and the jejunum 19 and on to the ileum and large intestines (not shown).

The small intestine is the longest section of the digestive tube and consists of three segments forming a passage from the pylorus to the large intestine: the duodenum, a short section that receives secretions from the pancreas and liver via the pancreatic and common bile ducts; the jejunum, which is generally considered to be roughly 40% of the small intestine in humans; and the ileum, which empties into the large intestine and is usually considered to be about 60% of the small intestine in humans.

Virtually all nutrients from the diet are absorbed into blood across the mucosa of the small intestine. By the time ingesta reaches the small intestine, foodstuffs have been mechanically broken down and reduced to a liquid by mastication and grinding in the stomach. The net effect of passage through the small intestine is absorption of most of the water and electrolytes (sodium, chloride, potassium) and essentially all dietary organic molecules (including glucose, amino acids and fatty acids). Through the activities, the small intestine not only provides nutrients to the body, but plays a critical role in water and acid-base balance.

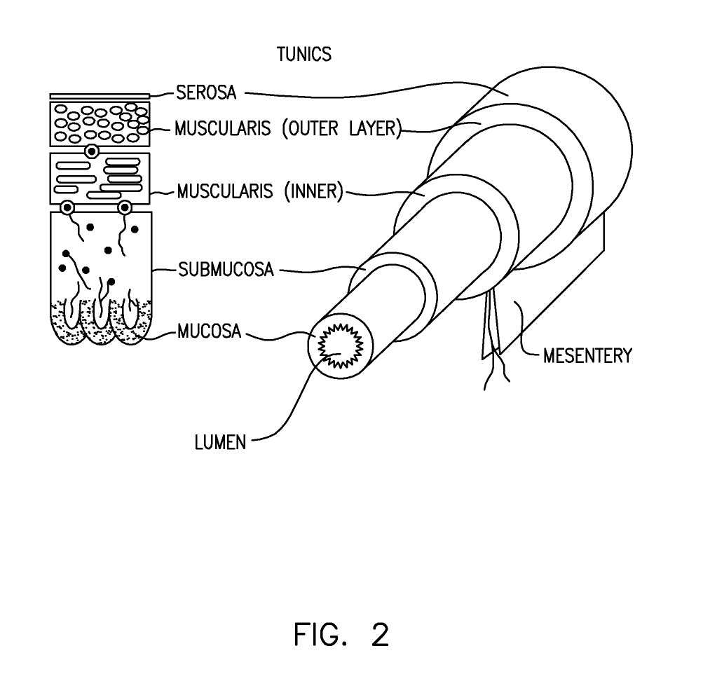

FIG. 2 is a schematic diagram of a portion of mammalian digestive tract, including the serosa, inner and outer muscularis, submucosa, mucosa and lumen. The tunica mucosa is the innermost layer of the digestive tube and lines the lumen. Among the four tunics, the mucosa is most variable in structure and function, endowing the tube with an ability to perform diverse and specialized digestive tasks along its length. Of critical importance in this regard are the epithelial cells that cover the mucosa and are thus in direct contact with the lumen. This epithelial cell sheet (lamina epithelialis) is distinctly different in different regions of the tract. Indeed, in most of the tract, several different cell types contribute to the epithelium, including cells dedicated to secretion, absorption or production of hormones. Beneath the epithelium, but still within the tunica mucosa is a layer—the lamina propria—of loose connective tissue through which course blood vessels and lymphatics that supply the epithelium. This layer also contains lymphatic nodules important to immune functions of the digestive tract.

The hormones most important in controlling digestive function are synthesized within the gastrointestinal tract by cells scattered in the epithelium of the stomach and small intestine. These endocrine cells and the hormones they secrete are referred to as the enteric endocrine system. The principal components of the enteric nervous system are two networks or plexi of neurons, both of which are embedded in the wall of the digestive tract and extend from esophagus to anus: the myenteric plexus is located between the longitudinal and circular layers of muscle in the tunica muscularis and exerts control primarily over digestive tract motility.

The submucous plexus is buried in the submucosa. Its principal role is in sensing the environment within the lumen, regulating gastrointestinal blood flow and controlling epithelial cell function. In regions where these functions are minimal, such as the esophagus, the submucous plexus is sparse and may actually be absent in sections. Motor neurons within the enteric plexuses control gastrointestinal motility and secretion, and possibly absorption. In performing these functions, motor neurons act directly on a large number of effector cells, including smooth muscle, secretory cells (chief, parietal, mucous, enterocytes, pancreatic exocrine cells) and gastrointestinal endocrine cells.

The gastrointestinal (GI) hormones are secreted by epithelial cells lining the lumen of the stomach and small intestine. These hormone-secreting cells—endocrinocytes—are interspersed among a much larger number of epithelial cells that secrete their products (acid, mucus, etc.) into the lumen or take up nutrients from the lumen. GI hormones are secreted into blood, and hence circulate systemically, where they affect function of other parts of the digestive tube, liver, pancreas, brain and a variety of other targets. The following is a brief discussion of some of the principle GI hormones.

Gastrin is released by G cells in the stomach and duodenum. The primary stimulus for secretion of gastrin is the presence of certain foodstuffs, especially peptides, certain amino acids and calcium, in the gastric lumen. Also, as yet unidentified compounds in coffee, wine and beer are potent stimulants for gastrin secretion. Secretion of this hormone is inhibited when the lumenal pH of the stomach becomes very low (less than about 3). Gastrin appears to have at least two major effects on gastrointestinal function, stimulation of gastric acid secretion and promotion of gastric mucosal growth.

Gastrin receptors are found on parietal cells, and binding of gastrin, along with histamine and acetylcholine, leads to fully-stimulated acid secretion by those cells. Enterochromaffin-like (ECL) cells also bear gastrin receptors, and recent evidence indicates that this cell may be the most important target of gastrin with regard to regulating acid secretion. Stimulation of ECL cells by gastrin leads to histamine release, and histamine binding to H2 receptors on parietal cells is necessary for acid secretion.

Gastrin has the ability to stimulate many aspects of mucosal development and growth in the stomach. Treatment with gastrin stimulates DNA, RNA and protein synthesis in gastric mucosa and increases the number of parietal cells. Another observation supporting this function is that humans with hypergastrinemia (abnormally high blood levels of gastrin) consistently show gastric mucosal hypertrophy.

In addition to parietal and ECL cell targets, gastrin also stimulates pancreatic acinar cells via binding to cholecystokinin receptors, and gastrin receptors have been demonstrated on certain populations of gastric smooth muscle cells, supporting pharmacologic studies that demonstrate a role for gastrin in regulating gastric motility.

Cholecystokinin plays a key role in facilitating digestion within the small intestine. It is secreted from mucosal epithelial cells in the first segment of the small intestine (duodenum), and stimulates delivery into the small intestine of digestive enzymes from the pancreas and bile from the gallbladder. Foodstuffs flowing into the small intestine consist mostly of large macromolecules (proteins, polysaccharides and triglyceride) that must be digested into small molecules (amino acids, monosaccharides, fatty acids) in order to be absorbed. Digestive enzymes from the pancreas and bile salts from the liver (which are stored in the gallbladder) are critical for such digestion. Cholecystokinin is the principle stimulus for delivery of pancreatic enzymes and bile into the small intestine.

The most potent stimuli for secretion of cholecystokinin known to date are the presence of partially-digested fats and proteins in the lumen of the duodenum. An elevation in blood concentration of cholecystokinin has two major effects that facilitate digestion: release of digestive enzymes from the pancreas into the duodenum; and contraction of the gallbladder to deliver bile into the duodenum. Cholecystokinin is also known to stimulate secretion of bile salts into the biliary system.

Pancreatic enzymes and bile flow through ducts into the duodenum, leading to digestion and absorption of the very molecules that stimulate cholecystokinin secretion. Thus, when absorption is completed, cholecystokinin secretion ceases.

Secretin is secreted in response to acidification of the duodenum, which occurs most commonly when liquified ingesta from the stomach are released into the small intestine. The principal target for secretin is the pancreas, which responds by secreting a bicarbonate-rich fluid, which flows into the first part of the intestine through the pancreatic duct. Bicarbonate ion is a base and serves to neutralize the acid, thus preventing acid burns and establishing a pH conducive to the action of other digestive enzymes. A similar response to secretin is elicited by bile duct cells, resulting in additional bicarbonate being dumped into the small gut. As acid is neutralized by bicarbonate, the intestinal pH rises toward neutrality, and secretion of secretin is turned off.

Ghrelin is another digestive hormone. At least two major biologic activities have been ascribed to ghrelin: stimulation of growth hormone secretion and regulation of energy balance. Other effects of ghrelin include stimulating gastric emptying and having a variety of positive effects on cardiovascular function (e.g. increased cardiac output).

Motilin participates in controlling the pattern of smooth muscle contractions in the upper gastrointestinal tract. There are two basic states of motility of the stomach and small intestine: the fed state, when foodstuffs are present, and the interdigestive state between meals. Motilin is secreted into the circulation during the fasted state at intervals of roughly 100 minutes. These bursts of motilin secretion are temporarily related to the onset of “housekeeping contractions”, which sweep the stomach and small intestine clear of undigested material. Some studies suggest that an alkaline pH in the duodenum stimulates release of motilin.

The teachings of the present invention include in one embodiment applying stimulation signals in a manner that promotes or inhibits GI hormone secretion in the region of the small intestine with which the signal is associated.

The scope of the invention further encompasses a method of treating metabolic disorders such as but not limited to type 2 diabetes and/or obesity in a patient. The method includes applying an electrical stimulation signal to a region of the small intestine of a patient, for example, to at least one or more regions of the pyloric sphincter, duodenum or jejunum of the patient with biophysical stimulation whereby the hormone producing cells of the region(s) are modulated. This method may be applied when the one or more regions include the entire length of the digestive tract including the pyloric sphincter, duodenum and jejunum; or a portion thereof. The method may include stimulation applied via a signal applied to the serosa of the digestive tube or the mucosa; or both. It should be understood that the appropriate region of the small intestine to be stimulated, as well as the location of the devices employed to deliver the signal(s) for a given patient will be determined by the diagnostic determination of the medical professional.

In a further embodiment, stimulation of specific portions of the small intestine or nerves innervating the regions is applied to treat disorders relating to diseases or conditions associated with the gastrointestinal tract. For example, the free flow of bile into the gut when no food matter is present is a powerful stimulant of sensations of hunger. This is more dramatically exhibited in patients who have experienced a cholecystectomy, and/or a post cholecystectomy sphincterotomy. In these patients, hunger pains can reach significantly discomforting levels, waking them up in the middle of the night and all but requiring them to eat something in order to affect a subsidence of the pain. This additional food intake, followed by a return to sleep, can easily lead to obesity.

In fact, it has been proposed that patients who are obese, and who complain of a near constant hunger that drives them to eat, may suffer from low tonicity in the sphincter of Oddi that results in a constant flow of bile into the digestive tract, which has the effect of amplifying and accelerating the return of hunger pains after ingesting a meal. A method of reducing a patient's feelings of hunger and thereby affect weight loss that is consistent with the present invention, therefore, comprises applying an electrical stimulation signal to modulate hormone secretions of the duodenum to effect reduced bile flow from the patient's common biliary duct into the patient's digestive tract.

Similarly, this same effect may be generated by applying a stimulation signal to nerve fibers associated with the hormone secreting cells in the enteric endocrine system. The stimulation may any suitable stimulation such as electrical, either through non-invasive means or by implanted electrodes; temperature, mechanical or vibrational.

In a preferred embodiment, the energy or stimulation signal is applied with an electrical impulse generating device. A number of different devices may be employed in accordance with the subject invention to electrically modulate a target region in the small intestine of a patient. Such devices may be positioned directly on a targeted area, e.g., positioned directly on or adjacent a portion of the target region (e.g., one or more nerve fibers) such as an implantable device, or may be an external device (i.e., some or all of the device may be external to the subject).

An electrical impulse generating device typically includes an electrode, a controller or programmer and one or more connectors for connecting the electrode to the controller. In certain embodiments, more than one electrode may be employed. The electrodes may be controllable to provide output signals that may be varied in voltage, frequency, pulse width, current and intensity. The electrodes may provide both positive and negative current flow from the electrodes and/or is capable of stopping current flow from the electrodes and/or changing the direction of current flow from the electrodes. In certain embodiments, the electrodes have the capacity for variable output, linear output and short pulse width.

The energy source for the electrical output is provided by a battery or generator such as a pulse generator that is operatively connected to the electrode. The energy source may be positioned in any suitable location such as adjacent to the electrode (e.g., implanted adjacent the electrode), or a remote site in or on the subject's body or away from the subject's body in a remote location and the electrode may then be connected to the remotely positioned energy source using wires, e.g., may be implanted at a site remote from the electrode or positioned outside the subject's body in certain instances. Of interest are implantable generators analogous to a cardiac pacemaker.

The electrodes may be mono-polar, bipolar or multi-polar. In order to minimize the risk of an immune response triggered by the subject against the device and minimize damage such as corrosion and the like to the device from other biological fluids, etc., the electrode and any wires and optional housing materials are made of inert materials such as for example silicon, metal, plastic and the like. Suitable electrodes may be formed from Pt—IR (90%/10%), although other materials or combinations or materials may be used, such as platinum, tungsten, gold, copper, palladium, silver or the like.

Those skilled in the art will also recognize that a variety of different shapes and sizes of electrodes may be used. By way of example only, electrode shapes according to the present invention can include ball shapes, twizzle shapes, spring shapes, twisted metal shapes, annular, solid tube shapes or the like. Alternatively, the electrode(s) may comprise a plurality of filaments, rigid or flexible brush electrode(s), coiled electrode(s) or the like. Alternatively, the electrode may be formed by the use of formed wire (e.g., by drawing round wire through a shaping die) to form electrodes with a variety of cross-sectional shapes, such as square, rectangular, L or V shaped, or the like.

A variety of methods may be used to endoscopically or surgically implant the electrode on or adjacent at least a portion of target region as is well known in the art.

A controller or programmer is also typically included in an electrical impulse generating device. The programmer is typically one or more microprocessors under the control of a suitable software program. Other components of the programmer will be apparent to those of skill in the art, e.g., analog to digital converter, etc. The device is typically pre-programmed for desired parameters. In many embodiments the parameters are controllable such that the electrode signal may be remotely modulated to desired settings without removal of the electrode from its targeted position. Remote control may be performed, e.g., using conventional telemetry with an implanted electric signal generator and battery, an implanted radiofrequency receiver coupled to an external transmitter, and the like. In certain embodiments, some or all parameters of the electrode may be controllable by the subject, e.g., without supervision by a physician. For example, a magnetic signal may be employed. In such embodiments, one or more magnets may be employed such that upon bringing a magnet in proximity to or away from the power source such as a pulse generator, the magnet may be employed to interfere with the electronic circuitry thus modulating the power—either increasing or decreasing the power supplied depending on whether the magnet is brought in proximity or moved away from the power source.

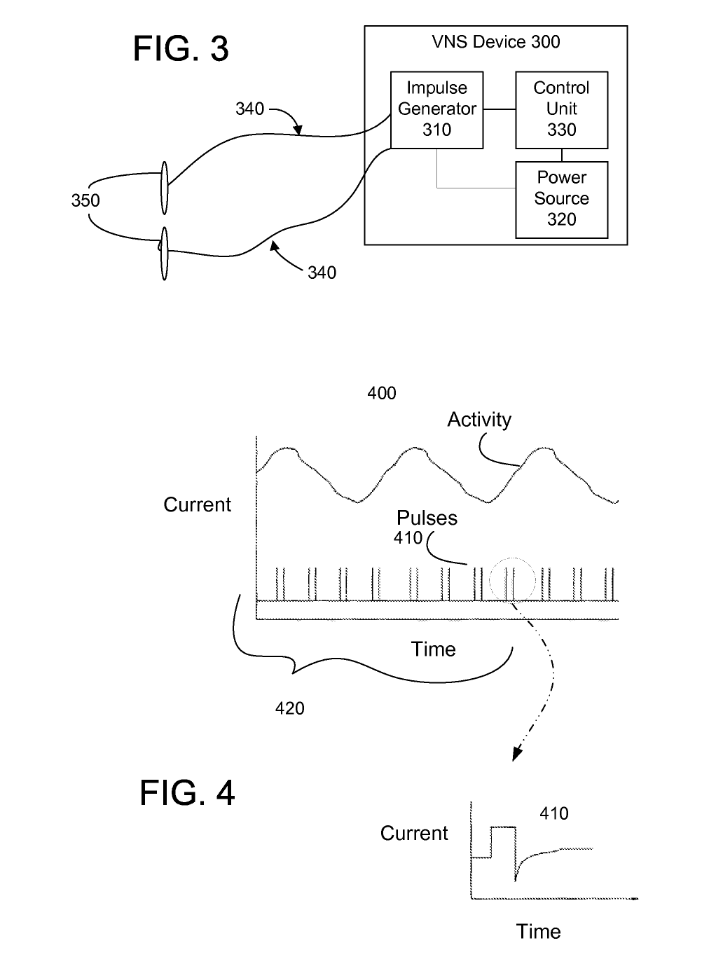

FIG. 3 is a schematic diagram of one embodiment of the present invention. As shown, an electrical impulse generating device 300 for delivering electrical impulses to nerves includes an electrical impulse generator 310; a power source 320 coupled to the electrical impulse generator 310; a control unit 330 in communication with the electrical impulse generator 310 and coupled to the power source 320; and an electrode assembly 340 coupled to the electrical impulse generator 310 for attachment via lead 350 to one or more selected regions of a nerve (not shown).

Device 300 may optionally include one or more sensors to provide closed-loop feedback control of the treatment therapy and/or electrode positioning. One or more sensors (not shown) may be attached to or implanted into a portion of a subject's body suitable for detecting a physical and/or chemical symptom or an important related symptom of the body. For example, sensing feedback may be accomplished, e.g., by a mechanical measure within a lead or an ultrasound or other sensor to provide information about the treatment parameters, lead positioning, etc.

The control unit 330 may control the electrical impulse generator 310 for generation of a signal suitable for amelioration of a patient's condition when the signal is applied via the electrode assembly 340 to the nerve. It is noted that nerve modulating device 300 may be referred to by its function as a pulse generator. U.S. Patent Application Publications 2005/0075701 and 2005/0075702, both to Shafer, both of which are incorporated herein by reference, relating to stimulation of neurons of the sympathetic nervous system to attenuate an immune response, contain descriptions of pulse generators that may be applicable to the present invention.

FIG. 4 illustrates an exemplary electrical voltage/current profile for a stimulating, blocking and/or modulating impulse applied to a portion or portions of selected nerves in accordance with an embodiment of the present invention. As shown, a suitable electrical voltage/current profile 400 for the blocking and/or modulating impulse 410 to the portion or portions of a nerve may be achieved using pulse generator 310. In a preferred embodiment, the pulse generator 310 may be implemented using a power source 320 and a control unit 330 having, for instance, a processor, a clock, a memory, etc., to produce a pulse train 420 to the electrode(s) 340 that deliver the stimulating, blocking and/or modulating impulse 410 to the nerve via lead 350. Nerve modulating device 300 may be powered and/or recharged from outside the body or may have its own power source 320. By way of example, device 300 may be purchased commercially such as the Itrel 3 Model 7425 available from Medtronic, Inc. Nerve modulating device 300 is preferably programmed with a physician programmer, such as a Model 7432 also available from Medtronic, Inc.

The parameters of the modulation signal 400 are preferably programmable, such as the frequency, amplitude, duty cycle, pulse width, pulse shape, etc. In the case of an implanted pulse generator, programming may take place before or after implantation. For example, an implanted pulse generator may have an external device for communication of settings to the generator. An external communication device may modify the pulse generator programming to improve treatment.

In addition, or as an alternative to the devices to implement the modulation unit for producing the electrical voltage/current profile of the stimulating, blocking and/or modulating impulse to the electrodes, the device disclosed in U.S. Patent Publication No.: 2005/0216062 (the entire disclosure of which is incorporated herein by reference), may be employed. U.S. Patent Publication No.: 2005/0216062 discloses a multi-functional electrical stimulation (ES) system adapted to yield output signals for effecting, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or a saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

The electrical leads 350 and electrodes 340 are preferably selected to achieve respective impedances permitting a peak pulse voltage in the range from about 0.2 volts to about 20 volts.

The stimulating, blocking and/or modulating impulse signal 410 preferably has a frequency, an amplitude, a duty cycle, a pulse width, a pulse shape, etc. selected to influence the therapeutic result, namely stimulating, blocking and/or modulating some or all of the transmission of the selected nerve. For example the frequency may be about 1 Hz or greater, such as between about 2 Hz to 100 Hz, more preferably between about 5 Hz to 50 Hz. The modulation signal may have a pulse width selected to influence the therapeutic result, such as about 10 ms or greater, preferably between about 20 μS to about 1000 μS. The modulation signal may have a peak voltage amplitude selected to influence the therapeutic result, such as about 0.2 volts or greater, such as about 0.2 volts to about 20 volts.

The mechanisms by which the appropriate stimulation is applied to the target tissue can include positioning the distal ends of an electrical lead or leads in the vicinity of a region of the small intestine, either on the outside or inside of the intestinal wall, which leads are coupled to an implantable or external electrical signal generating device. The electric field generated at the distal tip of the lead creates a field of effect that permeates the target tissue and cause the modulation of hormone release in the targeted region.

U.S. Pat. No. 6,928,320 to King describes the various frequency ranges that have been found to be effective for relaxing and activating various tissues. The specification of U.S. Pat. No. 6,928,320 and the references cited therein are incorporated by reference as examples of the various signal types that may be utilized to affect the therapeutic benefits encompassed by the present invention.

In addition, it is well known in the art that electrical field stimulation can induce gene expression. For example, pulsed electromagnetic fields (PEMF) are able to accelerate wound healing under diabetic and normal conditions by upregulation of FGF-2-mediated angiogenesis, See, Tepper et al., FASEB Journal express article 10.1096/fj.03-0847fje, (published online Jun. 18, 2004); Callaghan et al., Plast. Reconstr. Surg. 121:130-141 (2008); and PEMF has been shown to enhance osteogenic effects of BMP-2 on MSCs cultured on calcium phosphate substrates. Schwartz et al., J. Orthop Res. 1-8 (2008), published online in Wiley InterScience DOI 10.1002/jor.20591, all of the foregoing of which are incorporated herein by reference in their entireties.

In one embodiment stimulation is by way of a capacitively coupled (CC) electric field delivered via conducting electrodes placed on the outside or the inside of the intestinal wall. The signal is applied as a time varying sinusoidal voltage across the electrodes. The frequency of the signal may vary from 1 kHz to 100 kHz and may have a duty cycle from 1% to 100%. The amplitude of the signal is chosen so as to produce an electric field at the location of the device of from 0.1 to 100 mV/cm. In a preferred embodiment, the signal is a 60 kHz sine wave with amplitude of 20 mV/cm at the device.

In another embodiment, one or more flexible, stent-like coils is implanted in the lumen of the duodenum and/or another region of the small intestine, such as but not limited to the pyloric sphincter and jejunum, and an external electromagnetic device (tuned to the resonant frequency of the coil) is used to heat the coil several ° C. with a specific duty cycle to modulate the hormone releasing activity of the region in which the device is implanted so as to restore normal glucose metabolism.

In another embodiment, a mechanical signal is applied to the duodenum and/or other regions of the small intestine. A coil is placed within the duodenum and connected to an implanted mechanical actuator. Alternatively, a coil made of a temperature sensitive memory metal such as, but not limited to, nitinol is placed within the duodenum. An external device drives expansion and contraction of the coil by slight changes in temperature produced by an external electromagnetic field.

In another embodiment, a coil is connected to a direct current source which can modulate the pH of the duodenum and/or other regions of the small intestine through the release of acidic or basic Faradic Products.

The signal can consist of bursts of pulses repeated at a fixed frequency. The amplitude of each pulse can vary from 0.1 gauss to 50 gauss. The number of pulses/burst can range from 1 to 200. The burst frequency can range from 2 Hz to 100 Hz. The pulse duration can vary from 10 μs to 10 ms. In a preferred embodiment, the amplitude of the signal is 16 gauss, there are 20 pulses/burst, the bursts repeat at 15 Hz and during each pulse the magnetic field rises linearly to 16 gauss in 200 μs and then decays linearly to 0 in 25 μs.

Another form of stimulation employs an amplitude modulated radiofrequency signal. The signal is delivered by an external coil, similar to that used in an MRI machine, and the device acts as an antenna. The carrier frequency of the signal is such that its' wavelength in the tissue is twice the length of the device. This allows for maximal coupling of the signal to the device. For a typical device this corresponds to a carrier frequency of about 1-2 GHz. This carrier frequency may be modulated with waveforms as described above. In a preferred embodiment, the modulating waveform is a 5 ms long pulse repeating at 15 Hz.

In another embodiment, the device is made from a piezoelectric material. As intestinal pressure increases or decreases, the change in pressure produces a time varying potential difference across the device which stimulates a signal. Alternatively, the device may be coated with a thin electret material. The permanent electric field at the surface of the electret stimulates a signal.

In another embodiment, the device is coated with a material having a very high dielectric constant due to a high density of charged groups (similar to the glycocolyx surrounding a cell). As material flows past this layer, it generates a streaming potential which stimulates a signal.

In all cases, however, the implanting surgeon should vary the signal generated by the stimulation driver unit and specific location of the lead until the desired outcome is achieved, and should monitor the long-term maintenance of this effect to ensure that adaptive mechanisms in the patient's body do not nullify the intended effects.

Command(s) to the digestive system can be based on: (i) patient input (e.g., through wireless telemetry or magnet/reed switches) resulting from pain sensations or meal/bed time habits, etc.; (ii) responses to sensor data such as pressure in the patient's gall bladder or duct(s), nerve signals, stomach muscle signals, pH, concentration of enzymes and/or hormones; (iii) physician pre-programmed schedules; and/or (iv) a default software program in the stimulator.

The signals described above may be produced by an implanted generator or external stimulation device. The implanted generator may be powered and/or recharged from outside the body or may have its own power source. The signals may be electrical, vibrational or temperature-related. In one embodiment a vibrational device may be implanted that mimics peristalsis or creates a signal that is the same or similar to an electrical signal.

The signals to the digestive system may be applied with leads and electrodes, or the electrodes could be part of a leadless generator(s) attached to parts of the digestive system. An external stimulation device may use magnetic induction coil or coils, or pads attached to the skin. Sensor data may be sent to the implanted generator via wires or wireless communication. Sensor data to an external device is sent by wireless telemetry.

The implanted generator system may have an external device for communication of settings to the generator and/or information from the generator to the external device. The external communication device and/or generator/stimulation device may store sensor data and/or stimulation signals and timing information. These devices may have a computer interface to download data to the computer for analysis and trending. Such data could also be used to modify the generator/stimulator programming to improve treatment.

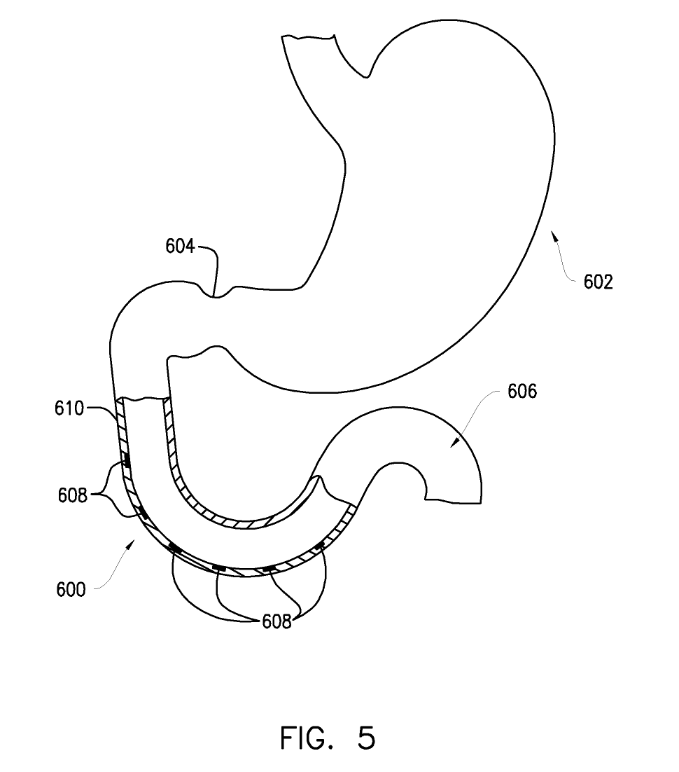

FIG. 5 schematically represents one embodiment of the present invention wherein multiple electrodes are implanted within the walls of a patient's duodenum 600. The duodenum 600 is the relatively short proximal or upper section of the small intestine that receives secretions from the pancreas and liver via pancreatic and common bile ducts (not shown). Gastric contents pass from the stomach 602 through the pylorus 604 to the duodenum 600 and then on to jejunum 606, the ileum and large intestines (not shown). It will be noted that the electrodes may also be implanted in either or both of the pylorus and the jejunum according to the present invention.

As shown, a series of electrodes 608 are implanted into the outer wall 610 of the duodenum 600. As described above, the enteric endocrine system is diffuse; i.e., hormone-secreting cells are scattered among other types of epithelial cells in the mucosa of the small intestine. Thus, a series of electrodes 608 are preferably implanted along the entire length of the duodenum 600 to optimize the effect of the electrical impulse on the cells within this region.

Electrodes 608 can be implanted in a variety of manners well known in the art. In certain embodiments, the electrodes 608 are configured to transmit an electrical field that will modulate the cells within the submocosa region of the duodenum wall. This region is responsible for sensing the environment within the lumen to control epithelial cell function. In this embodiment, sufficient energy is applied to the submocosa region to modulate the signals generated from this region, thereby controlling the release of hormones that may cause insulin resistance in certain patients. In other embodiments, electrodes 608 are configured to transmit an electrical field that will modulate the epithelial cells lining the lumen of the small intestine. These cells are responsible for actually secreting gastrointestinal hormones into the lumen of the GI tract. In this embodiment, sufficient energy is applied to these cells to modulate their production of hormones that may cause insulin resistance in certain patients.

The energy source (not shown) for delivering electrical impulses to electrodes 608 is provided by a battery or generator such as a pulse generator that is operatively connected to the electrodes 608. The energy source may be positioned in any suitable location such as adjacent to the electrode (e.g., implanted adjacent the electrode), or a remote site in or on the subject's body or away from the subject's body in a remote location.

In use, electrical impulses are delivered to electrodes 608 to modulate the hormone releasing activity of cells in the duodenum 600. In one embodiment, the electrical impulses are sufficient to inhibit the production of hormones that may cause insulin resistance in the patient. The inventors believe that inhibiting the production of these hormones or peptides will allow the patient to determine more normal concentrations of plasma glucose, insulin and glycosylated hemoglobin, thereby achieving increased glycemic control.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating or preventing type 2 diabetes in a patient, comprising applying energy to at least one region of the small intestine of the patient to modulate a hormone releasing activity of cells in the region.

2. The method of claim 1 wherein the cells are within the enteric endocrine system.

3. The method of claim 1 wherein the cells are nerves that modulate the enteric endocrine system.

4. The method of claim 1 the region is within at least one of the pyloric sphincter, duodenum and jejunum.

5. The method of claim 1 wherein the energy is sufficient to inhibit hormone production by the cells in the region.

6. The method of claim 1 wherein the energy is sufficient to stimulate hormone production by the cells in the region.

7. The method of claims 6 or 7 wherein the hormones are selected from the group consisting essentially of: secretin, cholecystokinin, gastrin, gastrin inhibitory protein, nitric oxide, vasoactive intestinal peptide, glucagon-like peptide, peptide YY, ghrelin, motilin, and other incretins and “anti-incretins”.

8. The method of claim 1 further comprising positioning at least one electrode in the region of the small intestine and applying an electrical impulse to the electrode.

9. The method of claim 8 wherein the region is a portion of the small intestinal wall.

10. The method of claim 1 wherein the energy is electrical.

11. The method of claim 1 wherein the energy is vibrational.

12. The method of claim 1 wherein the energy is temperature.

13. The method of claim 1 further comprising positioning at least one electrode on an outer surface of the patient's skin and applying an electrical impulse through the electrode to the region of the small intestine.

14. The method of claim 1 further comprising positioning a coil within the region of the small intestine and applying sufficient energy to the coil to modulate the hormone releasing activity of the cells.

15. The method of claim 14 wherein the applying energy step is carried out by applying electromagnetic energy to the coil to heat the coil.

16. The method of claim 14 wherein the applying energy step is carried out by expanding and contracting the coil.

17. The method of claim 1 further comprising positioning one or more electrodes on or within the region of the small intestine and applying a capacitively coupled electric field to the region through the electrodes.

18. The method of claim 1 further comprising positioning a coil outside of the patient's body and delivering an amplitude modulated radiofrequency signal to the region through the coil.

19. The method of claim 1 further comprising positioning a piezoelectric device within the region of the small intestine, wherein the piezoelectric device is adapted to produce a time varying potential difference to modulate the hormone releasing activity of the cells within the region.

20. A method for treating obesity in a patient comprising applying energy to at least one region of the small intestine of the patient to modulate hormone secretions, the energy being sufficient to effect reduced bile flow from the common biliary duct of the patient.

21. The method of claim 20 wherein the region is the duodenum.

22. The method of claim 20 wherein the region comprises nerve fibers associated with hormone secreting cells in the enteric endocrine system of the patient.

23. The method of claim 20 wherein the applying energy step comprises applying an electrical impulse to through one or more electrodes to the region.

24. A system for treating type 2 diabetes in a patient comprising:
a source of electrical energy;
one or more electrodes coupled to the source of electrical energy and adapted for applying an electrical impulse to a region of the small intestine of the patient; and
wherein the electrical impulse is sufficient to
modulate a hormone releasing activity of cells in the region.

25. The system of claim 24 wherein the region is within one of the pyloric sphincter, duodenum and jejunum.

26. The system of claim 24 wherein the electrical impulse is sufficient to inhibit hormone production by the cells in the region.

27. The system of claim 24 wherein the electrical impulse is sufficient to stimulate hormone production by the cells in the region.

28. The system of claim 24 wherein the hormones are selected from the group consisting essentially of: secretin, cholecystokinin, gastrin, gastrin inhibitory protein, nitric oxide, vasoactive intestinal peptide, glucagon-like peptide, peptide YY, ghrelin, motilin, and other incretins and “anti-incretins”.
